(19) United States
(12) Patent Application Publication
(10) Pub. No.: US 2004/0067250 A1
(43) Pub. Date: Apr. 8, 2004

(54) Biodegradable polystyrene capsules and manufacturing method thereof

(76) Inventors: Kyu-Teck Han, Seoul (KR); Jung-Hoon Choi, Seoul (KR); In-Gyu Lee, Kyunggi-do (KR); Ik-Soo Chung, Daejeon (KR)

Correspondence Address: Craig F Taylor, Fredrikson & Byron, 4000 Pillsbury Center, 200 South Sixth Street, Minneapolis, MN, 55402-1425, US

(21) Appl. No.: 10/466,790

(22) PCT Filed: Jan. 17, 2002
(86) PCT No.: PCT/KR02/00077

(30) Foreign Application Priority Data
Jan. 18, 2001 (KR) 2001/2992
Oct. 25, 2001 (KR) 2001/66085

(51) Int. Cl. A01N 59/06; A61K 33/10; A61K 9/48

(52) U.S. Cl. 424/451; 424/687

(57) ABSTRACT

The present invention relates to a biodegradable polystyrene capsule for molded product of polystyrene foam and to a method for manufacturing the same. The inventive biodegradable polystyrene capsule comprises a powder formed of a biodegradable material and a coating layer of foamable polystyrene resin formed on the surface of the powder. Products made from the biodegradable polystyrene capsule may variously used for packing material, insulating material, disposable products, etc. since the dame have great mechanical and physical properties such as impact-resistant property, anti-breakability, etc., and, since the biodegradable material inside the capsule is decomposed by microbes in the nature after a certain period, the efficiency of destruction is considerably improved, so it is possible to minimize problems of soil, air, and sea pollution caused by fill-in or incineration of the conventional wastes of molding foam product.

### BACKGROUND OF THE INVENTION

(a) Field of the Invention

The present invention relates to a biodegradable polystyrene capsules for molding product of polystyrene foam and to a manufacturing method thereof. More particularly, it relates to biodegradable polystyrene capsules as materials for molded product of polystyrene foam, which can minimize the conventional problems of soil, air, and sea pollution caused by fill-in or incineration of conventional wastes of molded foam product because physical properties such as impact-resistant property, anti-breakability, etc. of the inventive material are superior, and the biodegradable material in the capsules is decomposed by microbes in the natural world after a certain period, so the efficiency of destruction is considerably improved, and it also relates to a manufacturing method thereof.

(b) Description of the Related Art

Synthetic polymers represented by plastic are ones of the materials necessary for convenient and comfortable present-day life along with metals and ceramics. Such synthetic polymers are used for products of various industrial fields such as daily life material, construction, medical service, agriculture, etc. and the amount of use is considerably increasing. However, contrary to natural polymers, most of synthetic polymers are not easily decomposed, so the disposal and management of wastes of synthetic polymer products are big social problems for all the countries over the world.

Especially, molded products polystyrene foam made of EPS (Expandable Polystyrene) has good isolating properties against heat, noise, or humidity and great impact absorbency because it contains many independent air bubbles. Accordingly, such products are variously used as materials for keeping warmth or cold, packing material for absorbing shock from outside, insulating material, floating material, disposable products, etc. However, since wastes of such products have large volumes, a vast area is required for burying the wastes thereof. In addition, such products are so slowly decomposed, and the soil is polluted by the same. And if the wastes of the same flow to the sea, the sea can be polluted. When the wastes are destructed by fire, many poisonous gases are emitted to pollute the air.

Accordingly, some countries such as U.S.A. or Italy passes a bill to restrict the use of synthetic polymer products such as molded products of polystyrene foam which have a short period of use and which require durability not so much, and to substitute degradable material for them.

As a part of researches for overcoming these problems of disposal of synthetic polymer products, many recycling methods of wastes of styrene foam products are proposed.

For example, a method of recycling the wastes of styrene foam products by melting the same is disclosed in the Korean Pat. Laid-Open Publication No. 2000-59032 and a method of using the wastes of styrene foam products as reproducing material by reducing the same to a state of particles is disclosed in the Korean Pat. Publication No. 10-258635. However, such methods of recycling the molded foam products have no economical efficiency because they cost too high. In addition, the final wastes of the products should be disposed by the conventional methods such as burying in the end, and another environmental problems may occur during the recycling process.

### SUMMARY OF THE INVENTION

Accordingly, an object of the present invention is to overcome the above-mentioned problems and to provide biodegradable polystyrene capsules as materials for molded product of polystyrene foam, which can minimize the problems of soil, air, and sea pollution caused by fill-in or incineration of conventional wastes of molded foam product because physical properties of the inventive material such as impact-resistant property, anti-breakability, etc. are superior, and the biodegradable material in the capsules is decomposed by microbes in the natural world after a certain period, so the efficiency of destruction is considerably improved.

Another object of the present invention is to provide a method for manufacturing a biodegradable polystyrene capsule.

To achieve the object mentioned above, the present invention provides a biodegradable polystyrene capsule comprising a powder made from the biodegradable material and a coating layer of foamable polystyrene resin formed on the surface of the powder.

According to the biodegradable polystyrene capsule of the present invention, a coating layer of calcium alginate gel or a coating layer of alkylated calcium alginate gel may firstly be formed on the surface of the powder made from the biodegradable material before forming the coating layer of foamable polystyrene resin.

According to the biodegradable polystyrene capsule of the present invention, the grain may be used for the degradable powder forming core part of the capsule, especially, it is preferable to use corn powder, foamed corn powder, rice powder, and foamed rice powder.

According to the biodegradable polystyrene capsule of the present invention, when a coating layer of calcium alginate gel or alkylated calcium alginate gel is formed on the surface of the powder made from a biodegradable material, enzyme, microbe, animal cell, or plant cell may be added to regulate biodegradability and property of capsule. In addition, a binder such as polyvinyl alcohol, sodium alginate, gua gum, Arabic gum, or latex may be added to improve applicability of a coating layer of foamable polystyrene resin.

Further, to achieve the object mentioned above, the present invention provides biodegradable polystyrene capsules each comprising a capsule of calcium alginate gel containing carbon dioxide therein and a coating layer of foamable polystyrene resin formed on the surface of said capsule.

According to the biodegradable polystyrene capsules of the present invention, calcium alginate gel may be alkylated to control the degradability of capsules and to improve the coating property of foamable polystyrene resin.

To achieve the another object mentioned above, the present invention provides a method for manufacturing the biodegradable polystyrene capsules comprising steps of a) manufacturing capsules formed with a coating layer of calcium alginate gel on the surface of the powder by dropping an aqueous solution of sodium alginate in which the powder made from the biodegradable material is dispersed into an aqueous solution of calcium chloride while agitating; b) separating and drying the capsules; and c) forming a coating layer of foamable polystyrene resin on the surface of the separated capsules.

According to the method of the present invention, after the b) step, a step of alkylating the surface of the capsule by reacting the separated capsule with R—X(R is one selected from the group consisting of benzyl, ethyl, propyl, and isopropyl and X is one selected from the group consisting of chlorine, bromine, and iodine) may be added.

According to the method of the present invention, the step c) of forming a coating layer of foamable polystyrene resin may comprise steps of putting the separated capsule into a solution formed by dissolving the polystyrene resin in methylene chloride, evaporating methylene chloride, and impregnating one or more hydrocarbons having low boiling point selected from the group consisting of ethane, propane, butane, pentane, hexane, and octane at the condition of high temperature and high pressure. In addition, the step c) may be accomplished by putting the separated capsules into the suspension solution comprising one or more hydrocarbons having low boiling point selected from the group consisting of ethane, propane, butane, pentane, hexane, and octane and styrene monomer, and then performing the suspension polymerization.

According to the method for manufacturing biodegradable polystyrene capsules of the present invention, when a coating layer of foam polystyrene resin is formed, binder such as polyvinyl alcohol may be added to the solution formed by dissolving polystyrene resin in methylene chloride to improve applicability of the coating layer.

In addition, to achieve the another object mentioned above, the present invention provides a method for manufacturing the biodegradable polystyrene capsules comprising steps of a) manufacturing capsules formed with a coating layer of calcium alginate gel containing carbon dioxide therein while agitating, and dropping an aqueous solution formed by mixing sodium alginate with sodium bicarbonate into an aqueous solution of calcium chloride; b) separating and drying the capsules; and c) forming a coating layer of foamable polystyrene resin on the surface of the separated capsules. As mentioned above, after step b), a step of alkylating the surface of the capsules by reacting the separated capsules with R—X(R is one selected from the group consisting of benzyl, ethyl, propyl, and isopropyl, and X is one selected from the group consisting of chlorine, bromine, and iodine) may be added.

### DETAILED DESCRIPTION OF THE PREFERRED EMBODIMENTS

The detailed description about biodegradable polystyrene capsules and a method for manufacturing the same according to the present invention is provided hereinafter.

Degradable polymer is generally classified into biodegradable one, hydrolyzable one, photodegradable one, and oxidizable one according to the decomposition process. According to the U.S.A. ASTM definition, biodegradable one is the polymer decomposed by the microbes such as bacteria, fungi, and algae and hydrolyzable one is the polymer decomposed by the hydrolysis. Further, photodegradable one is the polymer decomposed by natural light, especially ultraviolet rays and oxidizable one is the polymer decomposed by oxidation. On the other hand, Biodegradable Plastic Society of Japanese defines the biodegradable polymers as the molecules having high molecular weight that could be decomposed into small molecules having low molecular weight by the microbes in the nature not to be harmful to the environment.

Accordingly, for being a biodegradable polymer, the material should be completely decomposed into water and carbon dioxide by the microbes in the nature and be returned to the nature not to make environmental problems. However, the microbe has substrate-specific properties. That is, a microbe has high reactivity to the compounds of specific molecular structure. Accordingly, though a synthetic polymer is designed to be biodegradable, it may not be effectively decomposed by the microbes which exist in the nature. Therefore, the present invention provides biodegradable capsules that may easily be decomposed by the microbes in the nature by using natural powder such as corn powder or natural polymer, i.e. alginic acid obtained from plants for core part of the foamable polystyrene resin.

Each of biodegradable polystyrene capsules according to an embodiment of the present invention is comprised of a powder made from the biodegradable material and a coating layer of foamable polystyrene resin formed on the surface of said powder.

Since the powder made from the biodegradable material is decomposed by microbes after a certain period, the coating layer of polystyrene resin formed on the surface thereof is destroyed. Accordingly, when the wastes of the products prepared by these biodegradable capsules are buried, their volume become remarkably smaller as time goes by, and the disposal efficiency of the wastes is considerably improved. For the biodegradable powder consisting the core part of a biodegradable capsule, all kinds of material may be used only if the same is biodegradable and the surface thereof may be coated with polystyrene resin. It is preferable to use inexpensive grain powder such as corn powder, foamed corn powder, rice powder, and foamed rice powder. Preferably, the average particle diameter of the biodegradable powder is 1 to 10 mm. As foamable polystyrene resin for coating the surface of the powder, various kinds of polystyrene resin known to those skilled in the art, for example, not only polystyrene resin but also various kinds of polystyrene resins being improved in quality such as block copolymer resin of styrene with butadiene, blend of these copolymers with polystyrene resin, and high impact polystyrene (HIPS) or else may be used(referring to Korean Pat. Laid Open Publication No. 2000-57292). On the other hand, a binder such as polyvinyl alcohol, sodium alginate, gua gum, Arabic gum, or latex may be added to improve applicability of the coating layer of foam polystyrene resin.

Each of biodegradable polystyrene capsules according to another embodiment of the present invention is comprised of the powder made from the biodegradable material, a coating layer of calcium alginate gel formed on the surface of said powder, and a coating layer of foamable polystyrene resin formed on the surface of said coating layer of calcium alginate gel.

Alginic acid, material for manufacturing calcium alginate gel that forms the layer of the biodegradable capsule according to the present invention may be obtained from the brown algae of oceanic plants in a large amount. Alginic acid is copolymer of straight chains of which the block of manuronic acid(M) unit, the block of gluronic acid(G) unit, and the block of MG unit, i.e. middle of M and G are composed with 1,4-glycoside and its molecular weight is 20,000˜200,000 or so. Alginic acid forms a gel by reacting with metallic ions such as calcium, and the gel is not melted by heat, so heat treatment is possible. Especially, since soft gel can be prepared owing to the M block, the property of the gel can be changed in accordance with the ratio of M/G. If the encapsulation is accomplished by adding enzyme, microbe, animal cell, or plant cell in the course of gelation, the biodegradability could be regulated.

As mentioned above, the coating layer of the calcium alginate gel formed on the surface of the biodegradable powder has great biodegradability and good elasticity, so it is possible to improve much more the physical properties of impact-resistance and anti-breakability. In addition, especially, if the calcium alginate gel on the surface of the capsule is alkylated by reacting the same with R—X(R is one selected from the group consisting of benzyl, ethyl, propyl, and isopropyl and X is one selected from the group consisting of chlorine, bromine, and iodine), the coating property of polystyrene resin can be improved much more. Moreover, there's an advantage that the degradability may be controlled by humidity since the hydrophobicity is changed according to the degree of alkylation.

Referring to a manufacturing method of such biodegradable polystyrene capsules, first, a capsule having a coating layer of calcium alginate gel on the surface of powder is made by dropping the aqueous solution of sodium alginate in which powder formed of biodegradable material such as foam corn powder is dispersed into an aqueous solution of calcium chloride while agitating. Here, the particle diameter of the capsule may be regulated according to the agitating speed. That is, when the agitating speed is high, the particle diameter is small and when the agitating speed is low, the capsule has relatively large particle diameter. It is preferable to agitate at the speed of 50 to 150 rpm. Next, the capsule is filtered with a filter or a centrifugal machine and then dried. In case that the surface of the coating layer of calcium alginate gel is alkylated, the dried capsule is put into methylene chloride and then the alkylation compound such as pyridine and benzyl chloride is added to obtain the capsule having a coating layer of alkylated calcium alginate gel.

After the capsule having a coating layer of calcium alginate gel is put into a solution formed by dissolving polystyrene resin in a solvent such as methylene chloride while agitating, and then the solvent is removed by vacuum distillation to obtain a capsule having a coating layer of polystyrene resin on the surface of the capsule. The coating layer of polystyrene resin may be formed by a general method such as a coating method of spraying a solution in which polystyrene resin is dissolved. Next, when the hydrocarbon having low boiling point such as ethane, propane, butane, pentane, hexane, and octane is impregnated under the condition of heat and pressure (preferably 50 to 95° C., and 3 to 10 bar), the foamability can be obtained.

In addition, the method of acquiring a coating layer of foamable polystyrene resin on the surface of the capsule having a coating layer of calcium alginate gel may be accomplished by putting the capsule into suspension solution comprising one or more hydrocarbon having low boiling point such as ethane, propane, butane, pentane, hexane, and octane and styrene monomer and performing the suspension polymerization instead of the method described above.

Then, products having desired use and shape can be manufactured by putting the biodegradable polystyrene capsules thus obtained above in a mold and foaming the same by spraying steam of high temperature.

Each of biodegradable polystyrene capsules according to another embodiment of the present invention comprises a capsule made of calcium alginate gel containing carbon dioxide inside the capsule and a coating layer of foam polystyrene resin formed on the surface of the capsule. Products made from such biodegradable capsules have great biodegradability as well as a superior impact-resistant property and elasticity since inside of the capsules are filled with gas. Especially, if the calcium alginate gel on the surface of capsule is alkylated by reacting the same with R—X(R is one selected from the group consisting of benzyl, ethyl, propyl, and isopropyl and X is one selected from the group consisting of chlorine, bromine, and iodine) before forming a coating layer of polystyrene resin, the coating property of polystyrene resin can be improved more. Further, the degradability by humidity may be regulated since the hydrophobic nature is changed according to the degree of alkylation.

A method for manufacturing biodegradable polystyrene capsules according to another embodiment of the present invention is as follows.

An elastic capsule comprised of porous calcium alginate gel containing carbon dioxide inside it is formed by dropping an aqueous solution of mixture of sodium alginate and NaHCO₃ into an aqueous solution of calcium chloride while agitating. Here, the particle diameter of the capsule may be regulated according to the agitating speed. That is, if the agitating speed is high, the particle diameter is small and if the agitating speed is low, the capsule has relatively large particle diameter. It is preferable to agitate at the speed of 50 to 150 rpm. After the capsule is filtered with a filter or a centrifugal machine and dried, the same is put into a solution formed by melting polystyrene resin in a solvent such as methylene chloride and then, the solvent is removed through vacuum distillation to obtain a capsule having a coating layer of polystyrene resin on the surface of the capsule. Next, if the hydrocarbon having low boiling point such as ethane, propane, butane, pentane, hexane, and octane is impregnated at high temperature and pressure, the foaming property can be obtained. In addition, the foaming property may be obtained by putting the capsule into the suspension solution comprising one or more hydrocarbons having low boiling point such as ethane, propane, butane, pentane, hexane, and octane and styrene monomer and then, performing suspension polymerization as described above.

[Embodiment]

The detailed description of the present invention referring to the embodiments is provided hereinafter. However, the embodiments according to the present invention can be modified in various ways and should not be understood to be restricted to the embodiments described below. The embodiments of the present invention are provided to describe the present invention more clearly to a person who has standard knowledge in the art.

Embodiment 1

Foamed corn grains are pulverized into particles having 2.5 mm of average particle diameter, 30.0 g of pulverized foamed corn powder is put into a solution made by dissolving 12.8 g of polystyrene in 22.0 ml of methylene chloride while agitating, and, 42.8 g of capsules having a coating layer of polystyrene resin on the surface of the corn powder is obtained by performing the vacuum distillation. 42.9 g of foamable capsules obtained by impregnating 4.3 ml of pentane into the above result at 80° C. and 10 bar are put in a molding foam device, and a product of the biodegradable polystyrene foam is manufactured by spraying 100° C. of steam.

Embodiment 2

Foamed corn grains are pulverized into particles having 2.5 mm of average particle diameter, 30.0 g of the pulverized foamed corn powder and an aqueous solution made by dissolving 0.1 g of polyvinyl alcohol in 0.5 ml of water are put into a solution made by dissolving 12.8 g of polystyrene in 22.0 ml of methylene chloride while agitating, and, 43.0 g of capsules having a coating layer of polystyrene resin and polyvinyl alcohol on the surface of the corn powder is obtained by performing the vacuum distillation. 43.1 g of foam capsules obtained by impregnating 4.3 ml of pentane into the above result at 80° C. and 10 bar are put in a molding foam device, and a product of biodegradable polystyrene foam is manufactured by spraying 100° C. of steam.

The physical properties of the molded products obtained according to the embodiments 1 and 2 are measured and described in following Table 1.

<A Method for Measuring the Physical Properties>

Biodegradability: measured according to the guide of OECD 301,C,MITI TEST(II)(1992).

Absorbing amount of moisture: measured according to a test method of KSM 3808.

Heat conductivity: measured according to a test method of KSM 3808.

Compressive strength: measured according to a test method of KSM 3808.

Flexural strength: measured according to a test method of KSM 3808.

TABLE 1

| | | Absorbing | | | |
|---|---|---|---|---|---|
| | | amount of | Heat | Compressive | Flexural |
| | Biodegradability | moisture | conductivity | strength | strength |
| | (%) | (g/100 cm²) | (kcal/m · hr · ° C.) | (kgf/cm²) | (kgf/cm²) |
| Embodiment 1 | 70 | 0.81 | 0.04 | 1.95 | 3.50 |
| Embodiment 2 | 71 | 0.92 | 0.03 | 2.08 | 3.81 |

Referring to Table 1, the molded products manufactured with the biodegradable polystyrene capsules according to the embodiments 1 and 2 have great biodegradability and good physical properties such as compressive strength, flexural strength, etc.

### MANUFACTURING EXAMPLE OF A COATING LAYER OF CALCIUM ALGINATE GEL

### Manufacturing Example 1

4.0 g of sodium alginate and 50.0 g of foam corn powder are added into a mixed solution of 20.0 ml acetone and 80.0 ml water at room temperature and the mixture is agitated for an hour. Next, the mixture is dropped into a saturated calcium chloride solution of 60° C. while agitating at 100 rpm to obtain 150.0 g of the porous biodegradable capsule coated with the calcium alginate gel on the surface of the corn powder.

### Manufacturing Example 2

4.0 g of sodium alginate, 50.0 g of foam corn powder, and 1 g of sodium bicarbonate are added into 100.0 ml of water at room temperature and the mixture is agitated for an hour. Next, the mixture is dropped into a saturated calcium chloride solution of 35° C. while agitating at 100 rpm to obtain 155.0 g of the porous biodegradable capsule coated with the calcium alginate gel on the surface of the corn powder.

### Manufacturing Example 3

4.0 g of sodium alginate and 1 g of sodium bicarbonate are added into 100.0 ml of water at room temperature and the mixture is agitated for an hour. Next, the mixture is dropped into a saturated calcium chloride solution of 30° C. while agitating at 100 rpm to obtain 10 g of the elastic capsule comprised of the calcium alginate gel containing carbon dioxide inside the capsule.

The physical properties of the molded products obtained according to the manufacturing examples 1 to 3 are measured and described in following Table 2.

<A Method of Measuring the Physical Properties>

Biodegradability: measured according to the guide of OECD 301,C,MITI TEST(II)(1992).

TABLE 2

| | | Thickness of coating | |
|---|---|---|---|
| | Average particle | layer of caicium | |
| | diameter of capsule | alginate gel | Biodegrad- |
| | (mm) | (mm) | ability (%) |
| Manufacturing | 2 | 0.008 | 97 |
| example 1 | | | |
| Manufacturing | 2 | 0.011 | 98 |
| example 2 | | | |
| Manufacturing | 2 | 0.010 | 99 |
| example 3 | | | |

Referring to Table 2, the capsules obtained according to the manufacturing examples 1 to 3 have a superior biodegradability and uniform thickness of coating layers.

Embodiment 3

70.0 g of the porous capsule obtained in the manufacturing example 1 and an aqueous solution made by dissolving 0.1 g of polyvinyl alcohol in 0.5 ml of water are put into a solution made by dissolving 30.0 g of polystyrene in 50.0 ml of methylene chloride while agitating at room temperature for an hour, and 100.0 g of the capsule having a coating layer of polystyrene resin and polyvinyl alcohol on the surface of the porous capsule is obtained by performing the vacuum distillation. 100.1 g of foamable capsules obtained by impregnating 10.0 ml of pentane into the above result at 80° C. and 10 bar are put in a molding foam device and a product of biodegradable polystyrene foam is manufactured by spraying 100° C. of steam.

Embodiment 4

30.0 g of the porous capsule obtained in the manufacturing example 1, is put into 90.0 ml of methylene chloride while agitating at room temperature. Next, 4.8 ml of pyridine and 6.9 ml of benzyl chloride are added to the mixture mentioned above and is agitated for 5 hours. Then, the capsules are separated, washed by water, and dried at 45° C. for 2 hours to obtain 31.0 g of capsules having hydrophobic nature. The obtained capsule is put into a solution made by dissolving 13.3 g of polystyrene in 22.1 ml of methylene chloride while agitating. Then, 44.3 g of capsule having a coating layer of polystyrene resin on the surface of it is obtained by performing the vacuum distillation. 44.4 g of foam capsule obtained by impregnating 4.4 ml of pentane into the above result at 80° C. and 10 bar for 30 minutes is put in a molding foam device and a product of biodegradable polystyrene foam is manufactured by spraying 100° C. of steam.

The physical properties of the products according to the embodiments 3 and 4 are measured by the same method as that of the embodiment 1 and described Table 3.

TABLE 3

| | | Absorbing | | | |
|---|---|---|---|---|---|
| | | amount of | Heat | Compressive | Flexural |
| | Biodegradability | moisture | conductivity | strength | strength |
| | (%) | (g/100 cm²) | (kcal/m · hr · ° C.) | (kgf/cm²) | (kgf/cm²) |
| Embodiment 3 | 78 | 0.71 | 0.04 | 2.01 | 3.70 |
| Embodiment 4 | 71 | 0.65 | 0.03 | 2.02 | 3.75 |

Referring to Table 3, the molded products manufactured with the biodegradable polystyrene capsules according to the embodiments 3 and 4 have great biodegradability and good physical properties such as compressive strength, flexural strength, etc.

As described above, since the products manufactured by the biodegradable polystyrene capsules have great mechanical and physical properties such as impact-resistant property, anti-breakability, etc., and the same may variously be used for packing material, insulating material, disposable products, etc. and since biodegradable material inside the capsules are decomposed by microbes in the nature after a certain period, the efficiency of destruction is considerably improved to minimize the problems of soil, air, and sea pollution caused by fill-in or incineration of conventional wastes of the molding foam product.

While the present invention has been described in detail with reference to the preferred embodiments, those skilled in the art will appreciate that various modifications and substitutions can be made thereto without departing from the spirit and scope of the present invention as set forth in the appended claims.

What is claimed is:

1. A biodegradable polystyrene capsule comprising a powder made from a biodegradable material and a coating layer of foamable polystyrene resin formed on the surface of said powder.

2. A biodegradable polystyrene capsule comprising a powder made from a biodegradable material, a coating layer of calcium alginate gel formed on the surface of said powder, and a coating layer of foamable polystyrene resin formed on the surface of said coating layer of calcium alginate gel.

3. A biodegradable polystyrene capsule comprising a powder made from a biodegradable material, a coating layer of alkylated calcium alginate gel formed on the surface of said powder, and a coating layer of foamable polystyrene resin formed on the surface of said coating layer of alkylated calcium alginate gel.

4. The biodegradable polystyrene capsule according to any one of claims 1 to 3, wherein said powder is made from grain.

5. The biodegradable polystyrene capsule according to claim 4, wherein said grain is one or more selected from the group consisting of corn, foamed corn, rice, and foamed rice.

6. The biodegradable polystyrene capsule according to claim 2, wherein said coating layer further comprises enzyme or microbe.

7. The biodegradable polystyrene capsule according to any one of claims 1 to 3, wherein said coating layer of foamable polystyrene resin further comprises binder.

8. The biodegradable polystyrene capsule according to claim 7, wherein said binder is one or more selected from the group consisting of polyvinyl alcohol, soda alginate, gua gum, Arabic gum, and latex.

9. A method for manufacturing a biodegradable polystyrene capsule comprising steps of:
a) manufacturing a capsule having a coating layer of calcium alginate gel on the surface of said powder by dropping an aqueous solution of sodium alginate in which a powder made from a biodegradable material is dispersed into a aqueous solution of calcium chloride while agitating;
b) separating and drying said capsule; and
c) forming a coating layer of foamable polystyrene resin on the surface of said separated capsule.

10. The method according to claim 9, wherein a step of alkylating the surface of the capsule by reacting the separated capsule with R—X(R is one selected from the group consisted of benzyl, ethyl, propyl, and isopropyl and X is one selected from the group consisted of chlorine, bromine, and iodine) is further comprised after the step b).

11. The method according to claim 9 or claim 10, wherein said step c) of forming a coating layer of foamable polystyrene resin comprises steps of putting said separated capsule in a solution made by dissolving polystyrene resin in methylene chloride, evaporating methylene chloride, and impregnating one or more hydrocarbons having low boiling point, selected from the group consisting of ethane, propane, butane, pentane, hexane, and octane, at high temperature and pressure.

12. The method according to claim 9 or claim 10, wherein said step of forming a second coating layer of foamable polystyrene resin in said step c) comprises steps of putting said separated capsule into suspension solution comprising one or more hydrocarbons having low boiling point selected from the group consisting of ethane, propane, butane, pentane, hexane, and octane and styrene monomer, and then performing the suspension polymerization.

13. The method according to claim 11, wherein said solution made by dissolving polystyrene resin in methylene chloride further comprises binder.

14. A biodegradable polystyrene capsule comprising a capsule formed of calcium alginate gel containing carbon dioxide inside the capsule and a coating layer of foamable polystyrene resin formed on the surface of said capsule.

15. A biodegradable polystyrene capsule comprising a capsule formed of alkylated calcium alginate gel containing carbon dioxide inside the capsule and a coating layer of foamable polystyrene resin formed on the surface of said capsule.

16. A method for manufacturing a biodegradable polystyrene capsule comprising steps of:
a) forming a capsule of calcium alginate gel containing carbon dioxide inside the capsule by dropping a mixed aqueous solution of sodium alginate and sodium bicarbonate into an aqueous solution of calcium chloride while agitating;
d) separating and drying said capsule; and
e) forming a coating layer of foamable polystyrene resin on the surface of the capsule.

17. The method according to claim 16, wherein a step of alkylating the surface of the capsule by reacting the separated capsule with R—X(R is one selected from the group consisting of benzyl, ethyl, propyl, and isopropyl and X is one selected from the group consisting of chlorine, bromine, and iodine) is further comprised after the step b).
